(19) United States
(12) Patent Application Publication
(10) Pub. No.: US 2009/0186350 A1
(43) Pub. Date: Jul. 23, 2009

(54) Muitiforms suspension microgranular bioreactor and methods of use

(76) Inventors: Gang Guo, Tianjin (CN); Rui Zhang, Tianjin (CN); Jingyu Zhang, Tianjin (CN); Dongchun Liang, Tianjin (CN); Baoli Wang, Tianjin (CN); Bei Sun, Tianjin (CN)

Correspondence Address: Limin Wen, Nichols Building 1st Floor, One Bungtown Road, Cold Spring Harbor, NY, 11724, US

(21) Appl. No.: 12/221,051

(22) Filed: Jul. 30, 2008

(30) Foreign Application Priority Data
Jan. 21, 2008 (CN) PCT/CN2008/000149

(51) Int. Cl. C12Q 1/68; C40B 40/00; C12M 1/34

(52) U.S. Cl. 435/6; 506/13; 435/287.2

(57) ABSTRACT

A Multiform Suspension Microgranular Bioreactor (MSMB) is used to detect nucleic acids or peptides/proteins in molecular and biological samples. Polymer-based microgranules, coupled with biomass molecule probes, are constructed with different features. The biomass molecule probes include cDNA probes, oligonucleic acid probes, peptide or protein probes. These microgranules are differentiated according to their features including shape, size, color, fluorescence intensity, magnetic property, gravity, chemical luminal intensity, radioactivity, and other labels. The above microgranules are suspended in a sample solution and are used collectively as a microarray-like bioreactor device. Such MSMB-based microarrays are particularly useful in complicated biological detection assays with flow cytometry.

### REFERENCE TO RELATED APPLICATION

This application is a national stage filing of International Application No. PCT/CN2008/000149 filed on Jan. 21, 2008. The teaching of the entire referenced application is incorporated herein by reference.

### FIELD OF THE INVENTION

The invention relates to the field of chemical and biological analysis and may be used for development of highly sensitive analytical devices for qualitative and quantitative analysis of molecular and biological samples including nucleic acids and peptides or proteins.

### BACKGROUND OF THE INVENTION

The following description provides a summary of information relevant to the present invention. It is not an admission that any of the information provided herein is prior art to the presently claimed invention or that any of the publications specifically or implicitly referenced are prior art to the invention.

Genomic, proteomic, and transcriptomic analyses of organisms have been increasingly conducted in medical and biotechnological fields. Vital activities are diverse and complicated phenomena that cannot be expressed as a simple combination of chemical reactions. However, a variety of and a large quantity of molecular information must be obtained so as to come closest to the actual natures of such vital phenomena. Accordingly, biological chips and assay systems therefore have been practically used. The biological chips include DNA chips and protein chips for evaluating the sequence and expression of genes in a collective manner. An example of these biological chips can be found in U.S. Pat. No. 5,744,305. Regular DNA chips each comprise a plate-like substrate and oligonucleotides having predetermined nucleotide sequences as one of DNA probes, in which the oligonucleotides are immobilized as spots at appropriate intervals on the substrate.

Although efforts to evaluate gene activity and to explain biological processes including those of disease processes and drug effects have traditionally focused on genomics in the past two decades, more attention has been paid to proteomics in recent years due to its offering a more direct, complete and promising understanding of the biological functions of a cell. Proteomics research is targeted towards a comprehensive characterization of the total protein complement encoded by a particular genome and its changes under the influence of biological perturbation. Proteomics also involves the study of non-genome encoded events such as the post-translation modification of proteins, interactions between proteins, and the location of proteins within the cell. The study of the gene expression at the protein level is important because many of the most important cellular activities are directly regulated by the protein status of the cell rather than the status of gene activity. Also, the protein content of a cell is highly relevant to drug discovery and drug development efforts since most drugs are designed to target proteins.

Attempts have been made to apply protein chips to various models such as the receptor-ligand relationship, antigen-antibody relationship, enzyme-substrate relationship, and agent-molecular point of action relationship. In many of these attempts, the probes are arranged two-dimensionally on a plane or are spread one-dimensionally, and their reaction conditions, complicated measuring procedures, and evaluation methods are still susceptible to improvements. Assay systems in the field of biology frequently include all the mixing system for mixing the sample and the reagent using an automatic sampler, the conveying system by the action of line control, and the assay system. Accordingly, these assays require a large amount of the sample, expensive and complicated instruments, and large amounts of reagents, and yield large amounts of wastes.

Recently, a chip-based proteomics approach has been introduced using biomolecular interaction analysis-mass spectrometry (BIA-MS) in rapidly detecting and characterizing proteins present in complex biological samples at the low- to sub-fmole level (Nelson et al., 2000 Electrophoresis 21: 1155-63). One of the most powerful techniques is surface enhanced laser desorption/ionization-time of flight-mass spectrometry (SELDI-TOF-MS) technology, which was commercially embodied in Ciphergens's ProteinChip Array System (Merchant et al., 2000 Electrophoresis 21: 1164-77). The system (aluminum chip) uses chemically (cationic, anionic, hydrophobic, metal, etc.) or biochemically (antibody, DNA, enzyme, receptor, etc.) treated surfaces for specific interaction with proteins of interest, followed by selected washes for SELDI-TOF-MS detection. However, the SELDI-TOF-MS based Protein Chip system suffers from the inability to provide the primary sequencing and structure information for biopolymers such as proteins and peptides, and for small compounds. It has limitations with respect to the quantitative analysis of analytes. It also has a limited detection level for analytes and limited range of proteins, since only a low number density of analyte is available at any small point on an array spot where the laser beam can hit and generate ions for detection. The detection levels will significantly decline for proteins with a molecular mass above 15-20 Kda.

With the ability of large-scale detection, biological chips became a popular research tool. However, the hybridization of nucleic acids and the interaction between proteins cannot be fully performed due to the effect of space hindrance with the gene probes and protein probes fixed in the solid-phase plane hybrid. This leads to high variation and low accuracy of the experimental results, and limits the applications of such biological chips.

There is a huge demand for the use in highly sensitive analytical devices for qualitative and quantitative analysis of molecular and biological samples and for design of a sensor provides for simplification and costs reduction; widening range of samples analyzed, improvement of their kinetic characteristics and increase in analysis sensitivity.

Microspheral chips come out to solve the problem of space hindrance. Currently, there are four types microspheral chips: 1) Antibody fluorescein-labeled microspheres by which the interaction is detected through the single-cell channel of the flow cytometry; 2) oligonucleic acids-labeled microspheres by which the interaction is detected through the single-cell channel of the flow cytometry; 3) magnetic materials-labeled microsperes by which the interaction is detected through the single-cell channel of the flow cytometry; 4) semiconductor nanocrystal microsperes by which the interaction is detected with 10 nm wave inducing different nanocrystals. All the above microspheral chips require first and second antibody, and different types of fluorescein.

However, all the above four types of microspheral chips cannot be interchanged between nucleic acid and protein. Also, the operation processes are quite complicated with different detection methods. Moreover, except for the nanocrystal microspheral chip that can be used to reach 10⁶ level theoretically, other types of microspheres cannot be labeled with more than 100 samples, which is lack of the characteristics of the large-scale detection in microarray. Since they have lost the characteristics of the original biological chips, they should not be regarded as biological chips any more.

In an effort to overcome the above space hindrance problem and keep large-scale characteristics and other biological chip's features, the present invention provides a microchip-based Multiform Suspension Microgranular Bioreactor (MSMB) to qualitatively and quantitatively detect molecular and biological samples including nucleic acids and peptides or proteins. This invention relates to devices and methods for performing active, multi-step molecular and biological sample detection and analysis.

### SUMMARY OF THE INVENTION

The present invention relates to a novel microarray made from a Multiform Suspension Microgranular Bioreactor (MSMB). Polymer-based microgranules are produced with biomass molecule probes coupled on them. The biomass molecule probes include cDNA probes, oligonucleic acid probes, peptide or protein probes. The above microgranules, constructed with different features, are suspended in a sample solution and are used collectively as a bioreactor to detect nucleic acids and/or peptides/proteins in the molecular and biological samples. After the interactions between probes and samples are completely and sufficiently performed, the microgranules are separated according to their features or forms through the single-cell channel of flow cytometry. Then, the interactions are measured qualitatively and quantitatively through an UV detector with 254 nm and 280 nm wavelength for nucleic acid and protein respectively.

This invention involves three processes: construct multiform microgranules with different features, separate microgranules according to their features, and analysis the detection through an UV detector. The key part of this invention is to prepare multiform microgranules and separate these microgranules according to their different features. There are basically nine different features disclosed in this invention, i.e. suspended microgranules can be sorted by shapes (sphere, tetrahedron, cube), size, color (RGB color system), color of fluorescence and fluorescence intensity, magnetic property, gravity, chemical luminal intensity, radioactivity, and other labels. The sorting of microgranules can be characterized into two stages: pre-processing and single-channel flow analysis. During the pre-processing stage, the microgranules are grouped by their size, gravity, and magnetic property. In the single-channel flow analysis stage, the microgranules are sorted by their colors, fluoresces, chemical luminance and radioactive labels. Such MSMB-based microarrays are particularly useful in complicated biological detection assays with flow cytometry. The advantages of MSMB-based microarrays include no space hindrance, large-scale detection capability, high specificity, great feature combination flexibility, high repeatability, and high-level automation.

### DETAILED DESCRIPTION OF THE PREFERRED EMBODIMENTS

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, cell and cancer biology, virology, immunology, microbiology, genetics and protein and nucleic acid chemistry described herein are those well known and commonly used in the art.

Construction of Multiform Microgranules

Polymers serve as the base to manufacture microgranules, on which the biomass molecules are fixed. Such biomass molecules could include, but not limited to, cDNA probes, oligonucleic acid probes, peptide or protein probes. The microgranules can be manufactured into following forms and with different features: 1) different shapes such as sphere, tetrahedron, and cube; 2) different sizes; 3) different colors (RGB color system); 4) different colors and intensities of fluorescence by adding fluorescent dyes; 5) different magnetic properties by adding magnetic materials; 6) different gravities by adding different ratios of heavy metal elements; 7) different chemical luminal intensities by adding different ratios of chemical luminal materials; 8) different wavelengths and intensities of radioactive rays by adding different ratios of radioactive nucleotides; 9) different labels such as by adding different ratios of biotins.

The selection of dyes and appropriate corresponding detection channels is well known and within the ability of one of skill in the art. Fluorescent dyes (fluorophores) suitable for use in the present invention can be selected from any of the many dyes suitable for use in imaging applications (e.g., flow cytometry). A large number of dyes are commercially available from a variety of sources, such as, for example, Molecular Probes (Eugene, Oreg.) and Exciton (Dayton, Ohio), that provide great flexibility in selecting a set of dyes having the desired spectral properties. Selection of candidate dyes can be carried out routinely based on the emission spectra of the dyes. Candidate dyes are then evaluated empirically by dyeing microgranules populations using a concentration series of each dye and subsequently analyzing the results. A suitable subset of the dyed microgranules are then selected for use together in a single array. Examples of fluorophores from which a suitable set can be selected include, but are not limited to, 4-acetamido-4′-isothiocyanatostilbene-2,2′ disulfonic acid; acridine and derivatives such as acridine, acridine orange, acrindine yellow, acridine red, and acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-amino-1-naphthyl)maleimide; anthranilamide; Brilliant Yellow; coumarin and derivatives such as coumarin, 7-amino-4-methylcoumarin (AMC, Coumarin 120), 7-amino-4-trifluoromethylcouluarin (Coumaran 151); cyanine and derivatives such as cyanosine, Cy3, Cy5, Cy5.5, and Cy7; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylaminocoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein isothiocyanate (FITC), fluorescein chlorotriazinyl, naphthofluorescein, and QFITC (XRITC); fluorescamine; IR144; IR1446; Green Fluorescent Protein (GFP); Reef Coral Fluorescent Protein (RCFP); Lissamine.™.; Lissamine rhodamine, Lucifer yellow; Malachite Green isothiocyanate; 4-methylumbelliferone; ortho cresolphthalein; nitrotyrosine; pararosaniline; Nile Red; Oregon Green; Phenol Red; B-phycoerythrin; o-phthaldialdehyde; pyrene and derivatives such as pyrene, pyrene butyrate and succinimidyl 1-pyrene butyrate; Reactive Red 4 (Cibacron.™. Brilliant Red 3B-A); rhodamine and derivatives such as 6-carboxy-X-rhodamine (ROX), 6-carboxyrhodamine (R6G), 4,7-dichlororhodamine lissamine, rhodamine B sulfonyl chloride, rhodamine (Rhod), rhodamine B, rhodamine 123, rhodamine X isothiocyanate, sulforhodamine B, sulforhodamine 101, sulfonyl chloride derivative of sulforhodamine 101 (Texas Red), N,N,N′,N′-tetramethyl-6-carboxyrhodamine (TAMRA), tetramethyl rhodamine, and tetramethyl rhodamine isothiocyanate (TRITC); riboflavin; rosolic acid and terbium chelate derivatives; xanthene; or combinations thereof. Other fluorophores or combinations thereof known to those skilled in the art may also be used, for example those available from Molecular Probes (Eugene, Oreg.) and Exciton (Dayton, Ohio). It will be clear to one of skill in the art that the suitability of particular dyes or classes of dyes will depend on the method by which the microgranules are labeled, as described further, below. For example, large fluorescent proteins may be suitable for labeling microgranules by binding the dyes to the surface of the microparticle, but likely would not be suitable for internal labeling using bath-dyeing methods. Suitable candidate dyes can be selected routinely based on the labeling methods used.

Fluorescent dyes have been incorporated into uniform microspheres in a variety of ways, for example by copolymerization of the fluorescent dye into the microspheres during manufacture (U.S. Pat. No. 4,609,689 to Schwartz et al. (1975), U.S. Pat. No. 4,326,008 to Rembaum (1982), both incorporated by reference); by entrapment of the fluorescent dye into the microspheres during the polymerization process; or by non-covalent incorporation of the fluorescent dye into previously prepared microspheres (U.S. Pat. Nos. 5,326,692; 5,723,218; 5,573,909; 5,786,219; and 6,514,295; each incorporated by reference). The method of labeling the microspheres is not a critical aspect of the invention; any method that allows the labeling of the microgranules with a controllable amount of dye can be used.

Methods for attaching an antibody or other target-specific reagent to a microgranule are known in the art. Commercially available microgranules typically are provided with amino groups or carboxyl groups to facilitate the covalent attachment of antibodies using well-known chemistry. However, any method used by those skilled in the art may be employed.

Separation of Nucleic Acids and Peptides/Proteins by Microgranules' Features

Flow cytometry is well known analytical tools that enable the characterization of particles on the basis of light scatter and particle fluorescence. In a flow cytometry, particles are individually analyzed by exposing each particle to an excitation light, typically one or more lasers, and the light scattering and fluorescence properties of the particles are measured. Particles, such as molecules, analyte-bound beads, individual cells, or subcomponents thereof, typically are labeled with one or more spectrally distinct fluorescent dyes, and detection is carried out using a multiplicity of photodetectors, one for each distinct dye to be detected. Flow cytometry is commercially available from, for example, BD Biosciences (San Jose, Calif.).

Early in the development of flow cytometry, it was recognized that various types of ligand binding assays could be carried out using microgranules (beads) coated with one member of a binding pair. For example, immunoassays can be carried out in a sandwich hybridization assay format using beads coated with an analyte-specific binding agent, such as a monoclonal antibody (mAb), as a capture reagent, and a second analyte-specific binding agent, again typically a mAb, labeled with a fluorophore as a reporter reagent. The coated beads and reporters are incubated with a sample containing (or suspected of containing) the analyte of interest to allow for the formation of bead-analyte-reporter complexes. Analysis by flow cytometry enables both detecting the presence of bead-analyte-reporter complexes and simultaneously measuring the amount of reporter fluorescence associated with the complex as a quantitative measure of the analyte present in the sample.

It was also recognized early in the development of flow cytometry that the simultaneous analysis of multiple analytes in a sample could be carried out using a set of distinguishable beads, each type of bead coated with a unique analyte-specific binding agent. The bead set and fluorescently labeled reporter reagents, one for each species of analyte to be detected, are incubated with a sample containing the analytes of interest to allow for the formation of bead-analyte-reporter complexes for each analyte present, and the resulting complexes are analyzed by flow cytometry to identify and, optionally, quantify the analytes present in the sample. Because the identity of the analyte bound to the complex is indicated by the identity of the bead, multiple analytes can be simultaneously detected using the same fluorophore for all reporter reagents. A number of methods of making and using sets of distinguishable microgranules have been described in the literature.

UK Patent No. 1 561 042, published Feb. 13, 1980, and Fulwyler and McHugh, 1990, Methods in Cell Biology 33:613-629, describe the use of multiple microgranules distinguished by size, wherein each size microparticle is coated with a different target-specific antibody. Tripatzis, European Patent No. 0 126,450, published Nov. 28, 1984 (see also corresponding Canadian Patent 1 248 873), describes multi-dimensional arrays of microgranules formed by labeling microgranules with two or more fluorescent dyes at varying concentrations. Microgranules in the array are uniquely identified by the levels of fluorescence dyes. Tripatzis describes the use of such arrays for the simultaneous detection a large numbers of analytes in a sample by flow cytometry, and, further, describes their use as labels in microscopy. U.S. Pat. Nos. 4,499,052 and 4,717,655, Entitled: “Method and Apparatus for Distinguishing Multiple Subpopulations of Cells”, issued Feb. 12, 1985, and Jan. 5, 1988, respectively, describe the use of microgranules distinguishably labeled with two different dyes, wherein the microgranules are identified by separately measuring the fluorescence intensity of each of the dyes. Both one-dimensional and two-dimensional arrays for the simultaneous analysis of multiple analytes by flow cytometry are available commercially. Examples of one-dimensional arrays of singly dyed beads distinguishable by the level of fluorescence intensity include the BD.™. Cytometric Bead Array (CBA) (BD Biosciences, San Jose, Calif.) and Cyto-Plex.™. Flow Cytometry microspheres (Duke Scientific, Palo Alto, Calif.). An example of a two-dimensional array of beads distinguishable by a combination of fluorescence intensity (five levels) and size (two sizes) is the QuantumPlex.™. microspheres (Bangs Laboratories, Fisher, Ind.). An example of a two-dimensional array of doubly-dyed beads distinguishable by the levels of fluorescence of each of the two dyes is described in McDade and Fulton, April 1997, Medical Device & Diagnostic Industry; and Fulton et al., 1997, Clinical Chemistry 43(9):1749-1756. Each of the microparticle arrays described above has disadvantages that limit their utility. One-dimensional arrays based on differences in the fluorescent intensity of a single dye typically are limited to about 10 different microparticle populations. Although useful for a wide range of assays, it is desirable to have more distinct microparticle populations to enable the simultaneous detection of greater numbers of analytes. Two-dimensional arrays based on differences in the fluorescence intensities of two distinct dyes enable much larger arrays, but are still not enough for the large-scale level in a microarray analysis.

The present MSMB-based microarrays are particularly used for detect molecular and biological samples such as nucleic acids and peptides/proteins. After all the interactions (including hybridizations) between probes and samples have been fully performed in the solution, it is essential to differentiate the probe-sample interacted microgranules. The current invention combines the following nine features to separate the probe-sample interacted microgranules: 1) sorting microgranules according to their shapes (sphere, tetrahedron, or cube) by using a detector; 2) sorting microgranules into several groups according to size by controlling the channel of flow cytometry and the detector; 3) sorting microgranules according to color (RGB color system) which has red, green, and blue serving as the basic colors by organizing them into groups by different combination methods and colors; 4) sorting microgranules according to the color and intensity of fluorescence by adding the exact quantity of two types of fluorescein (e.g. Red fluorescent) with different spectra into microgranules and characterizing microgranules into several groups by different ratios of that two fuorescein through spectra analysis classified by the color of fluorescence and fluorescence intensity (each group exhibits a measurably distinct fluorescence intensity); 5) sorting microgranules according to their magnetic properties by adding magnetic materials into microgranules and swing the magnetic particles absorbed with the microgranules under different magnetic field intensities (Polymer-based microgranules contain magnetic oxides and possess super paramagnets, which means under the outside magnetic field, microgranules can be quickly separated from the dispersing medium, and when re-entering the magnetic field, microgranules again become suspended in the medium, without the magnet); 6) sorting microgranules according to their specific gravity by adding different ratios of heavy metal elements into polymer-based microgranules and creating subgroups with different gravity with hybrid buffers; 7) sorting microgranules according to their chemical luminal intensity by adding different ratios of chemical luminal materials into microgranules and creating subgroups of different luminal intensity by inducing particles luminance; 8) sorting microgranules according to wavelength and intensity of radioactive rays by adding different ratios of radioactive nucleotides into microgranules and creating subgroups of different radioactive intensities with the different wavelengths; 9) sorting microgranules according to other labeling methods (e.g. biotin) by adding different ratios of biotins into microgranules and creating subgroups of different intensities by inducing particles luminance.

The above nine features can be applied collectively or with different combinations to produce polymer-based microgranules which can be grouped according to different criteria, then each microgranule possesses one feature to all features. For example, microgranules can be grouped by color using the third and fourth feature above into 20 subgroups and can be grouped by intensity into 10 subgroups; microgranules can be grouped using the first method above into 3 subgroups; each of all the other features above can be used to group microgranules into 10 subgroups; then the total number of all the possible subgroups will be up to 1.2×10¹¹ (3×10×20×10×20×10×10×10×10×10×10) which completely meets the need of large-scale analysis. The sorting of microgranules can be proceeded into two stages: pre-processing and single-channel flow analysis. During pre-processing stage, the microgranules are grouped by their size, gravity, and magnetic properties. In the single-channel flow analysis stage, the microgranules are sorted by their colors, fluoresces, chemical luminance and radioactive labels.

The Qualitative and Quantitative Analysis of Biological Samples Interacted with the Probes on the Microgranules

The suspended microgranules which possessing biomass molecules are sorted through single-channel flow cytometry. Using the UV detector, the absorbance is measured in 254 nm and 280 nm wavelength for detecting nucleic acids and proteins from samples. The results are statistically classified for qualitative and quantitative analysis.

Quantitative analysis steps: 1) Using the UV detector, measure the absorbance value in 254 nm and 280 nm wavelength of microgranules which possessing probes (nucleic acids or proteins/peptides) without interacting with any samples. The results serve as base values. 2) Using the UV detector, measure the absorbance value in 254 nm and 280 nm wavelength of the above microgranules after interacting with nucleic acids or proteins from samples, subtract the base value from the second measurements above, and conclude positive or negative interaction according to the base values. During the reactions, both positive and negative controls are added.

Qualitative analysis steps: 1) Using the UV detector, measure the absorbance value in 254 nm and 280 nm wavelength of microgranules which possessing probes (nucleic acids or proteins/peptides) without interacting with any samples. The results serve as base values. 2) Using the UV detector, measure the absorbance value in 254 nm and 280 nm wavelength of the above microgranules after interacting with standard concentrations of nucleic acids or proteins, then subtract the base value from the second measurements and establish related standard curve. 3) Using the UV detector, measure the absorbance value in 254 nm and 280 nm wavelength of the above microgranules after interacting with nucleic acids or proteins from samples, subtract the base values, and calculate the concentrations of hybridized nucleic acids or interacted peptides/proteins from samples according to the standard curve. During the reactions, both positive and negative controls are added.

The MSMB-based microarrays of the present invention can be used essentially in any application in which multiplex particle arrays are used or are useful, including applications in which the microgranules are used a solid substrates for ligand binding assays or as labeling reagents. For use in such microassays, the microgranules are coated with analyte-specific reagents such that microgranules within a population are coated with reagents having the same known specificity and microgranules in different populations are coated with reagents having different specificities. One skilled in the art will understand that detection can be carried out using any of a number of different assay formats, including sandwich hybridization formats and competitive assay formats.

The advantages of the MSMB-based microarrays are described as following:
No space hindrance for the interactions between probes and samples;
Large-scale analysis capability of detecting over 1.2×10¹¹ types of samples;
High specificity with hybridization or interaction condition;
Great flexibility with nine features under different combinations;
High repeatability with sufficient probe-sample interactions;
High-level automation through flow cytometry technology.

The practice of the various aspects of the present invention may employ, unless otherwise indicated, conventional techniques of chemistry, cell biology, cell culture, molecular biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Current Protocols in Molecular Biology, by Ausubel et al., Greene Publishing Associates (1992, and Supplements to 2003); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Lodish et al., Molecular Cell Biology, 4th ed., W. H. Freeman & Co., New York (2000); Griffiths et al., Introduction to Genetic Analysis, 7th ed., W. H. Freeman & Co., New York (1999); Gilbert et al., Developmental Biology, 6th ed., Sinauer Associates, Inc., Sunderland, Mass. (2000); and Cooper, The Cell—A Molecular Approach, 2nd ed., Sinauer Associates, Inc., Sunderland, Mass. (2000). All patents, patent applications and references cited herein are incorporated in their entirety by reference. Those skilled in the art will recognize, or be able to ascertain using no more than routine experimentation, many equivalents to the specific embodiments of the invention described herein.

### Definitions.

With the description of this invention, a number of terms used in are utilized extensively. In order to provide a clear and consistent understanding of the specification and claims, including the scope to be given such terms, the following definitions are provided for clarity. Unless otherwise indicated, all terms are used as is common in the art. All reference cited herein are incorporated herein by reference.

The term “analyte” is used herein broadly to refer to any substance to be analyzed, detected, measured, or labeled. Examples of analytes include, but are not limited to: proteins, peptides, hormones, haptens, antigens, antibodies, receptors, enzymes, nucleic acids, polysaccarides, chemicals, polymers, pathogens, toxins, organic drugs, inorganic drugs, cells, tissues, microorganisms, viruses, bacteria, fungi, algae, parasites, allergens, pollutants and combinations thereof. It will be understood that detection of, for example, a cell, is typically carried out by detecting a particular component, such as a cell-surface molecule, and that both the component and the bacteria as a whole can be described as the analyte.

As used herein, the term “microgranules” refers to small particles with a diameter in the nanometer to micrometer range, typically about 0.01 to 1,000 .mu.m in diameter, preferably about 0.1 to 100 .mu.m, more preferably about 1 to 100 .mu.m, and, for use in flow cytometry, typically about 1 to 10 .mu.m. Microgranules can be of any shape, but typically are sphere, tetrahedron, and cube. Microgranules serve as solid supports or substrates to which other materials, such as target-specific reagents, reactants, and labels, can be coupled. Microgranules can be made of any appropriate material (or combinations thereof), including, but not limited to polymers such as polystyrene; polystyrene which contains other co-polymers such as divinylbenzene; polymethylmethacrylate (PMMA); polyvinyltoluene (PVT); copolymers such as styrene/butadiene, styrene/vinyltoluene; latex; or other materials, such as silica (e.g., SiO.sub.2).

Microgranules suitable for use in the present invention are well known in the art and commercially available from a number of sources. Unstained microgranules in a variety of sizes and polymer compositions that are suitable for the preparation of fluorescent microgranules of the invention are available from a variety of sources, including: Bangs Laboratories (Carmel, Ind.), Interfacial Dynamics Corporation (Portland, Oreg.), Dynal (Great Neck, N.Y.), Polysciences (Warrington, Pa.), Seradyne (Indianapolis, Ind.), Magsphere (Pasadena, Calif.), Duke Scientific Corporation (Palo Alto, Calif.), Spherotech Inc. (Libertyville, Ill.) and Rhone-Poulenc (Paris, France). Chemical monomers for preparation of microspheres are available from numerous is sources.

As used herein, “microgranules population” refers to a group of microgranules that possess essentially the same optical properties with respect to the parameters to be measured, such as synthesized microgranules that, within practical manufacturing tolerances, are of the same size, shape, composition, and are labeled with the same kind and amount of dye molecules. For example, unlabeled microgranules, microgranules labeled with a first dye at a first concentration, microgranules labeled with the first dye at a second concentration, and microgranules labeled with a second dye at the third concentration could constitute four distinct microgranules populations.

### REFERENCES

1. Taylor J D, Briley D. et al. Flow cytometric platform for high-throughput singlenucleotide polymorphism analysis. Bio Techniques. 2001, 30:611-669
2. Spain M, Mcdade R. A workstation approach to bioassays. IVD Technol. 2000, 6:35-42
3. Cheng J, Fortina P, Sorrey S, et al. Microchip-based device fir molecular of genetic disease [J]. Molecular Diagnosis, 1996:(1), 183-190.
4. Burt J. P. H., Chan K. L., Dawson, D., Parton A., Pethig R. Assays for microbial contamination and DNA analysis based on electrorotation[J]. Ann. Biol. Clin, 1996, (54): 253-257.
5. Pohl H A. Dielectrophoresis[M]. Cambridge: Cam-bridge University Press, 1978.
6. Jones T B. Electromechamics of particles[M]. Cambridge: Cambridge University Press, 1995, 5-33.
7. Markx G H, Talary M S, Pethig R. Separation of viable and non-viable yeast using dielectroph-oresis [J]. J Biotechnology, 1994, 32(1):29-34.
8. Pethig R. Dielectrophoresis: using inhomogeneo-us AC electrical fields to separate and manipulate cells[J]. Crit. Rev. Biotechnology, 1996, (16):331-348.
9. Sanders G H W, Manz A. Chip-based Microsystems for genomic and proteomic analysis[J]. Treds Anal Chem, 2000, 19 (6):364-378.
10. Pethig R, Markx G. H. Applications of dielectrophoresis in biotech-nology[J]. Trends in Biotechnology, 1997, (15):426-432.
11. Pethig R. Dielectric and electronic properties of biological materials[M]. J. Wiley & Sons, 1979, 186-206.
12. Nagy G R, Ban Z, Sipos F, et al. First attempts of detecting fetal cells in the maternal circulation. Orv Hetil, 2004, 145:2 231-2 236.
13. Taubert H, Blumke K, Bilkenroth U, et al. Detection of disseminated tumor cells in peripheral blood of patients with breast cancer: correlation to nodal status and occurrence of metastases. Gynecol Oncol, 2004, 92:256-261.
14. Sakamoto A, Abe M, Masaki T. Delineation of genomic deletion in cardiomyopathic hamster. FEBS Lett, 1999, 447:124-128.
15. Nam J M, Thaxton C S, Mirkin C A. Nanoparticle-based bio-bar codes for the ultrasensitive detection of proteins. Science, 2003, 301:1 884-1 886.
16. van Helden J, Denoyel G, Freeman J, et al. Performance of a new HIV 1/O/2 assay on the Bayer ADVIA Centaur immunoassay system. Clin Lab, 2004, 50:83-90.
17. Tanaka T, Matsunaga T. Fully automated chemiluminescence immunoassay of insulin using antibody-protein A-bacterial magnetic particle complexes. Anal Chem, 2000, 72:3 518-3 522.
18. Jaiswal J K, Mattoussi H, Mauro J M, et al. Long-term multiple color imaging of live cells using quantum dot bioconjugates. Nat Biotechnol, 2003, 21:47-51.
19. Goldman E R, Clapp A R, Anderson G P, et al. Multiplexed toxin analysis using four colors of quantum dot fluororeagents. Anal Chem, 2004, 76:684-688.
20. Wang J, Polsky R, Xu D, et al. Silver-enhanced colloidal gold electrochemical stripping detection of DNA hybridization. Langmuir, 2001, 17:5 739-5 741.
21. Csaki A, Moller R, Straube W, et al. DNA monolayer on gold substrates characterized by nanoparticles labeling and scanning force microscopy. Nucleic Acids Res, 2001, 29:E81.

1. A multiform suspension microgranular bioreactor, comprising suspended microgranules attached with nucleic acid probes or protein probes.

2. The multiform suspension microgranular bioreactor of claim 1, wherein said suspended microgranules are made from polymers.

3. The multiform suspension microgranular bioreactor of claim 1, wherein said suspended microgranules are prepared and characterized with the following features: shape, size, color and intensity of color, color of fluorescence and intensity of fluorescence, magnetic property, gravity, chemical luminal intensity, radioactivity, and other labels.

4. The multiform suspension microgranular bioreactor of claim 1, wherein each of said nucleic acid probes or protein probes attached on said suspended microgranules is correspondent to a specific said feature in claim 3.

5. A large-scale detection method of nucleic acids and proteins is carried out through the multiform suspension microgranular bioreactor said in claim 1 to claim 4.

6. The large-scale detection method of claim 5, wherein said detection method is carried out by flow cytometry.

7. The large-scale detection method of claim 5, comprising a process to separate said suspended microgranules with two stages: pre-processing and single-channel flow analysis.

8. The large-scale detection method of claim 7, wherein said pre-processing stage is to separate said suspended microgranules according to size, gravity, and magnetic property.

9. The large-scale detection method of claim 7, wherein said single-channel flow analysis stage is to separate said suspended microgranules according to color, fluoresces, chemical luminance and radioactive labels.

10. The large-scale detection method of claim 5, wherein said nucleic acids and proteins are detected by measuring the absorbance values with UV detector in 254 nm and 280 nm wavelength for nucleic acid and protein respectively.

11. An array of said suspended microgranules of claim 1 to claim 4 for detecting nucleic acids and proteins in a sample.
